(19) United States
(12) Patent Application Publication
(10) Pub. No.: US 2011/0033512 A1
(43) Pub. Date: Feb. 10, 2011

(54) PERSONAL CARE COMPOSITION WITH IMPROVED SPREADABILITY

(76) Inventor: Laurie Ellen Breyfogle, Milford, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/851,921

(22) Filed: Aug. 6, 2010

Related U.S. Application Data
(60) Provisional application No. 61/232,614, filed on Aug. 10, 2009.

(51) Int. Cl. A61K 8/02; A61Q 17/04; A61K 8/92; A61K 9/00; A61P 29/00; A61Q 19/00

(52) U.S. Cl. 424/401; 424/59; 424/60; 424/484

(57) ABSTRACT

A stable personal care composition in the form of a substantially anhydrous oil matrix having improved spreadability that may comprise a hydrocarbon wax, a polar oil, and a non-emulsifying silicone elastomer. The weight ratio of the hydrocarbon wax to the polar oil is from about 0.01 to about 0.5. The personal care composition may exhibit a spreadability of 1.0 kg to about 5.0 kg and a hardness of about 3 g to about 120 g

### CROSS REFERENCE TO RELATED APPLICATION

This application claims the benefit of provisional application No. 61/232,614, filed Aug. 10, 2009.

### FIELD OF THE INVENTION

The present invention relates to a personal care composition in the form of an oil matrix comprising a hydrocarbon wax, a polar oil, and a non-emulsifying silicone elastomer with improved spreadability.

### BACKGROUND OF THE INVENTION

A variety of products provide protection to the skin and other keratinous tissue from the harmful effects of ultraviolet radiation (UV). Much work has been done on improving the efficacy and stability of products providing UV protection. However, attributes such as skin feel and ease of application of products providing UV protection have not been well investigated. Such aesthetics cannot be discounted because they have a direct impact on the efficacy of the product. A leading indicator of UV protection products is the sun protection factor (SPF) which is determined by standard methods with application of 2.0 mg/cm² of product to skin. Unfortunately, consumers are less rigorous in their application procedures of sun protection products, often applying too little product and/or failing to periodically reapply. This results in less UV protection than expected based on the SPF labeling. Consumers are far less likely to use the requisite 2.0 mg/cm² of product if the product has poor aesthetics. To encourage liberal and frequent use of UV protection products, it is important that the product have a desirable appearance and also a pleasant feel when applied.

Most highly effective sunscreen agents have a consumer disfavored heavy, oily skin feel. Personal care compositions comprising sunscreen agents can be formulated as emulsions to improve skin feel, but maintaining a stable emulsion while retaining required skin feel and UV protection is difficult. For example, oil-in-water emulsions, wherein the product is stabilized by using polymeric thickeners to thicken the aqueous phase (e.g., predominately water), are not consumer preferred due to aesthetic issues surrounding the heavy product feel during and after application thereof. Alternately, water-in-oil emulsions, wherein the oil phase is thickened/stabilized using fatty alcohol waxes have a tendency to feel greasy and heavy. Silicone elastomers may be used to counteract the poor aesthetics of either emulsion form, but silicone elastomers add further challenges to the formulation and are typically quite expensive. Silicone elastomer blends can impact sunscreen agent solubility (which directly impacts efficacy of UV protection if the sunscreen should precipitate) and overall product stability.

Personal care compositions may be in the form of an oil matrix such as an oil dispersion or oil solution. These forms are generally disfavored because it is difficult to mask the oily or greasy feel and options available for thickening such matrices are not are not abundantly available. Silicone elastomers may be used to both thicken the matrix and counteract the poor aesthetics, but, as with emulsions, silicone elastomers add further challenges to the formulation. Silicone elastomers can impact sunscreen agent solubility (which directly impacts efficacy of UV protection) and overall product stability. Additional the thickening capacity of silicone elastomers may be compromised in the presence of the more effective UV sunscreen actives. Solid sticks may be formed through the inclusion of waxes to enhance stability and mask some of the oily feel, but are not consumer preferred due to poor spreadability and aversion to the use of stick products on the full face. Spreadability is the ease with which the product may be spread into a thin layer, for example as one would spread a composition onto the skin, by applying shear force such as wiping.

The extent of UV protection offered by a product is also, in part, dependent on the uniformity of the product on skin. Due to the uneven topology of skin, sun protection products of a thin consistency tend to flow downward into the wrinkles of the skin leaving higher skin surfaces with lesser or no sunscreen coating. Thicker products are expected to achieve a more uniform sunscreen product film on skin and thus enhanced sun protection.

Another problem with existing UV formulations is balancing the UV protection against other consumer deliverables. Most consumer sunscreens are formulated and marketed such that sun protection is the sole or predominate purpose of the product. However, personal care products are formulated for other purposes such as for moisturization, anti-aging treatment and prevention, cleansing, and acute change (i.e., make-up). UV protection is added to these personal care products to provide a more holistic benefit to consumers, but the formulation challenges are heightened. When providing a product that achieves the primary goal of the personal care product such as a facial moisturizer or anti-aging treatment, aesthetics are critical. Consumers will not use a product regardless of efficacy if the aesthetics are not suitable (i.e., petrolatum is an excellent emollient but consumers will not accept the aesthetics of applying a greasy, tacky product to the face). Net, existing formulations may attempt to achieve a stable UV product but do not teach, discuss, or consider the requirements of aesthetics.

### SUMMARY OF THE INVENTION

A stable personal care composition may be provided in the form of a substantially anhydrous oil matrix comprising from about 1% to about 10% of a hydrocarbon wax, greater than about 10% of a polar oil, and from about 1% to about 30% of a first non-emulsifying silicone elastomer; wherein the weight ratio of wax to polar oil is from about 0.01 to about 0.5; wherein the personal care composition exhibits a spreadability of 1.0 kg to about 5.0 kg and a hardness of about 3 g to about 100 g.

Further aspects of the present invention will be appreciated in the detailed description that follows.

### DETAILED DESCRIPTION OF THE INVENTION

All percentages are by weight of the personal care composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements. All measurements are understood to be made at about 25° C. and at ambient conditions, where “ambient conditions” means conditions under about one atmosphere of pressure and at about 50% relative humidity.

“Personal care composition,” as used herein, means compositions suitable for topical application on mammalian keratinous tissue. Compositions of the present invention may be used in skin-care, cosmetic, and hair-care products; non-limiting uses of which include antiperspirants, deodorants, lotions (e.g. hand lotion and body lotion), skin-care products (e.g., face and neck lotions, serums, sprays), sunless tanners, cosmetics (e.g., foundation, concealer, blush, lipstick, lip gloss), depilatories, shampoos, conditioning shampoos, hair conditioners, hair dyes, body washes, moisturizing body washes, shower gels, skin cleansers, cleansing milks, hair and body washes, in-shower body moisturizers, pet shampoos, shaving preparations, after-shaves, razor moisturizing/lubricating strips, razor shave-gel bars, bar soaps, cleansing products, feminine-care products, oral-care products, and baby-care products. The methods of using any of the aforementioned compositions are also included within the meaning of personal care composition.

“Beauty Care Composition,” as used herein, means a personal care composition formulated with a skin care active, which is not a sunscreen, wherein the composition improves the condition of keratinous tissue. The beauty care composition may secondarily provide UV protection through the inclusion of a sunscreen. Conditions to be improved include increasing the luminosity or “glow” of the skin, reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); increasing skin or hair elasticity, for example, due to loss, damage and/or inactivation of functional skin elastin, resulting in such conditions as elastosis, sagging, loss of skin or hair recoil from deformation; reduction in cellulite; changing coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

“Keratinous tissue,” as used herein, refers to keratin-containing layers disposed as the outermost protective covering of mammals which includes, but is not limited to, skin, hair, and nails.

“Stable” and “stability,” as used herein, refer to compositions which exhibit no syneresis when left for 5 days at a controlled environmental temperature of approximately 21° C. and 40° C. The syneresis test is ideally conducted with samples of at least 50 g. Ideally samples are prepared by filling cylindrical containers with the dimensions of 1.5″ tall and 2.0″ in diameter with approximately 50 g of the composition. The sample is visually monitored at temperature to determine if liquid synereses from the bulk product over time. If no syneresis occurs at either temperature, the product is deemed “stable.” If syneresis occurs at either temperature, the stability is deemed “unstable

“Derivatives,” as used herein, means ester, ether, amide and/or salt derivatives of the relevant compound.

“Polar,” as used herein, means a material with a solubility parameter of greater than or equal to 7.4 (calories/cm³)^{0.5} to about 11 (calories/cm³)^{0.5}. “Non-polar,” as used herein, means a material with a solubility parameter of less than 7.4 (calories/cm³)^{0.5}. Solubility parameters are discussed in more detail by C. D. Vaughan in “The Solubility Parameter: What is it?,” Cosmetics & Toiletries vol. 106, November, 1991, pp. 69-72. Solubility parameter may be determined or calculated according to procedures discussed within Barton, AFM (1991), Handbook of Solubility Parameters and Other Cohesion Parameters, 2nd edition, CRC Press.

“Hardness,” as used herein, means the amount of force in grams (g) necessary for a cylindrical probe having a diameter of 2 mm (probe TA-52 from Texture Technologies) to penetrate a distance of 0.5 mm at a rate of 1 mm/s into the composition, using a TA-XT2i Texture Analyzer with Texture Expert Exceed software (v. 2.64). Ideally samples are prepared by filling cylindrical containers with the dimensions of 1.5″ tall and 2.0″ in diameter with approximately 50 g of the composition; comparative samples may be measured in the container as purchased. Prior to measuring the hardness, the composition is allowed to equilibrate to 21° C.+/−1° C.

“Solid,” as used herein, means a composition with a hardness of greater than or equal to 120 g as measured by a texture analyzer.

“Spreadability,” as used herein, refers to the ability of the composition to be spread into a thin layer, for example as one would spread a composition onto the skin, by applying shear force such as wiping. Spreadability may be determined by the following method, which would be understood by one of skill in the art. Using a TA-425TTC Spreadability Fixture, i.e. a male and a female acrylic 90° cone, having a height of 25 mm (Texture Technologies Corp.), fill the female cone with specified product using a spatula. Carefully avoid excessively working the product during the filling procedure; gently press the product down as much as needed to eliminate air pockets. Level the product surface with a straight-edge. Measure the maximum force in kilograms (kg) required for the male cone to traverse the product in the female cone, resulting in displacement of the product from the female cone, at a rate of 3 mm/s, until the distance between the lower portion of the male cone and the female cone is exactly 1.5 mm. Set the withdrawal speed to 10 mm/s. The temperature of the composition and cone apparatus is 21° C.+/−1° C.

To overcome the challenge of stabilizing an oil matrix in a personal care composition while maintaining formulation flexibility, the present invention relates to a product with a unique hardness overcoming the perception of greasiness while still maintaining desirable spreadability. While not wishing to be bound by theory, unlike solid products which contain a wax crystal network to trap the oil, the stability advantage obtained by the present invention holds regardless of the degree of connections between crystals, i.e. a crystal network is not necessary to contain the oil. This yields improved aesthetics allowing for the formation of products that exhibit improved hardness and structure while retaining consumer preferred attributes like ease of spreadability.

The personal care composition may be in the form of a substantially anhydrous oil matrix. The term “substantially anhydrous” means that water and hydrophilic components are not intentionally added to the matrix; however, small amounts of water hydrophilic components incidentally may be present, for example as a by-product of chemical reactions, or as a result of absorption from the environment. The personal care composition comprises at least about 15% of an oil. In certain embodiments, the personal care composition comprises from about 20% to about 90% of an oil phase, or from about 40% to about 90% of an oil phase. The oil phase may comprise silicone oils, non-silicone oils such as hydrocarbon oils, esters, ethers, the like, and mixtures thereof. In a one embodiment, the oil phase comprises silicone oils, polar hydrocarbon oils, and mixtures thereof.

Polar Oil—In one aspect, the personal care composition comprises a polar oil. The personal care composition may comprise greater than about 10% of polar oil or, in an alternate embodiment, greater than about 20% of polar oil. Suitable polar oils include ethers, esters, amides, propoxylates, and mixtures thereof. The aforementioned oils may be saturated, unsaturated, aliphatic (straight or branched chains), alicyclic, or aromatic.

Suitable polar oils include but are not limited to, butyl and isopropyl phthalimide (Pelemol™ BIP), phenylethyl benzoate (X-tend™ 226), dicaprylyl carbonate (Tegosoft™ DEC), isopropyl lauroyl sarcosinate (Eldew™ SL 205), butyl octylsalicylate (Hallbrite™ BHB), dioctyl malate, dicaprylyl maleate (Hallbrite™ DCM), di-isopropyl adipate, dibutyl adipate (Cetiol B), isononyl isononanoate, isopropyl isostearate, propylene glycol dicaprate, C12-15 alcohol benzoate (Finsolv TN), PPG-11 stearyl ether, and derivatives and mixtures thereof.

Other exemplary polar oils include retinoid such as retinol and retinol propionate, tocopherol (i.e., vitamin E), derivatives of tocopherol, and tetrahexyldecyl ascorbate.

Sunscreens are another example of polar oils. Exemplary sunscreens include but are not limited to, benzophenone-3, bis-ethylhexyloxyphenol methoxyphenyl triazine, butyl methoxydibenzoylmethane, diethylamino hydroxy-benzoyl hexyl benzoate, ethylhexyl triazone, drometrizole trisiloxane, ethylhexyl methoxy-cinnamate, ethylhexyl salicylate, octocrylene, homosalate, polysilicone-15, 2-ethylhexyl N,N-dimethyl-p-aminobenzoate, 4-isopropyl dibenzoylmethane, 3-(4-methylbenzylidene) camphor, 3-benzylidene camphor, and menthyl anthranilate. In one embodiment, the composition comprises more than one sunscreen.

In one embodiment, the polar oil comprises at least one oil-soluble sunscreen which, in its commercially-available purified form, is an oil-soluble crystalline and/or solid compound. It is to be understood that the oil-soluble crystalline and/or solid sunscreen is substantially dissolved, and thus does not remain in a crystalline form in the personal care composition of the present invention. For example, a suitable polar oil may comprise a combination of an oil-soluble crystalline sunscreen and a solvent such as butyl methoxydibenzoylmethane with isopropyl lauroyl sarcosinate. Particularly suitable crystalline sunscreens include benzophenone-3, bis-ethylhexyloxyphenol methoxyphenyl triazine, butyl methoxydibenzoylmethane, diethylamino hydroxy-benzoyl hexyl benzoate, and ethylhexyl triazone.

Hydrocarbon Wax—The composition comprises a hydrocarbon wax. “Hydrocarbon wax,” as used herein, means a substance that is solid at room temperature and consists of the elements carbon and hydrogen. The hydrocarbon wax is substantially free of heteroatoms (i.e. oxygen, nitrogen), as it is made from pure hydrocarbon reactants. Any heteroatom present in the hydrocarbon wax is an unintended impurity. The hydrocarbon structure may be straight, branched, or napthenic and may be derived synthetically or naturally. Sources include, but are not limited to, Fischer-Tropsch synthesis, alkene polymerization, petrolatum, and mineral. In certain embodiments, the personal care composition comprises from about 0.01% to about 10%, or from about 1% to about 8%, or from about 4% to about 6%, by weight of the composition, of a hydrocarbon wax.

Suitable hydrocarbon waxes include saturated hydrocarbons.

Suitable hydrocarbon waxes include straight-chain hydrocarbon waxes, as defined by having less than 5% CHR₃ polymeric subunits, wherein R is a hydrocarbon chain of length C₁-C₁₀₀, are preferred. Examples include, but are not limited to, polyethylene, polymethylene, and similar synthetic waxes. In some embodiments, the composition comprises more than one type of hydrocarbon wax.

In certain embodiments, the hydrocarbon wax may exhibit one or more of the following: a saturation of greater than about 90%; from 20 to 100 carbon atoms, or from 25 to 80 carbon atoms, or from 30 to 70 carbon atoms; have less than 5% CHR₃ polymeric subunits, wherein R is a hydrocarbon chain of length C₁-C₁₀₀; a breadth of molecular weight distribution or polydispersity index of less than about 1.1, or less than about 1.05, as defined by the weight-average degree of polymerization divided by the number-average degree of polymerization; a melt point of from about 55 degrees C. to about 150 degrees C., or from about 65 degrees C. to about 100 degrees C.; a needle penetration at 25 degrees C. as measured by ASTM 1321 of from about 1 dmm to about 45 dmm, or from about 2 dmm to about 15 dmm, or from about 2 dmm to about 10 dmm. The unit dmm designates tenths of a millimeter.

It has been found that if the weight ratio of wax to polar oil is too low, the composition becomes unstable and synereses. Conversely, if the weight ratio of wax to polar oil is too high, the composition takes the form of a solid stick and exhibits consumer disfavored properties such as poor spreadability. The personal care composition of the present invention exhibits a hardness at 21° C. from about 3 g to about 120 g; from about 3 g to about 100 g; from about 10 g to about 75 g; or from about 20 g to about 50 g. In solid sticks, a continuous wax crystal network forms thereby entrapping the oil. However, solid sticks are unappealing to consumers and have poor spreadability. Solid sticks feel noticeably waxy—e.g., heavy, less powdery soft, and slow absorbing. These negative aesthetics are particularly noticeable and highly undesirable when applied to the full face. The personal care composition of the present invention exhibits a spreadability of from about 1.0 kg to about 5.0 kg or from about 2.0 kg to about 4.0 kg. Accordingly, it has been found that a weight ratio of wax to polar oil of from about 0.01 to about 0.5, or from about 0.1 to about 0.5, or from about 0.2 to about 0.4 is suitable.

Non-emulsifying Silicone Elastomer—In one aspect, the personal care composition comprises a first non-emulsifying silicone elastomer. “Non-emulsifying silicone elastomer” means that the silicone elastomer comprises no polyoxyalkylene groups. The personal care composition may comprise from about 1% to about 30%, or from about 2% to about 20%, or from about 3% to about 10%, by weight of the composition, of a first non-emulsifying silicone elastomer.

The composition may comprise a second non-emulsifying silicone elastomer that is chemically different from the first non-emulsifying silicone elastomer. “Chemically different” means that the materials have unique International Nomenclature of Cosmetic Ingredients (INCI) name, which are available in the Personal Care Product Council's International Cosmetic Ingredient Dictionary and Handbook, 12^{th} Ed. and the 2008 International Buyers' Guide. The personal care composition may comprise from about 1% to about 10%, or from about 2% to about 8%, or from about 4% to about 6%, by weight of the composition, of a second non-emulsifying silicone elastomer.

Suitable non-emulsifying silicone elastomers include the INCI designated dimethicone/vinyl dimethicone crosspolymers such as supplied by Dow Corning™ (DC 9506, DC 9701, EP9261T1, EP9215, and EP9293AL), General Electric™ (SFE 839), Shin Etsu™ (KSG 15 and 16), and Grant Industries (GRANSIL™ RPS-NA) and dimethicone/phenyl vinyl dimethicone crosspolymer such as KSG 18 available from Shin Etsu™. Other exemplary silicone elastomers include the INCI designated dimethicone crosspolymers including Dow Corning™ DC 9040, DC 9041, and DC 9045. The non-emulsifying silicone elastomers may also be surface treated to include but not limited to silica, titanium dioxide, and alumina.

A non-emulsifying silicone elastomer may be supplied pre-swollen with a solvent (or may be swollen with a solvent prior to compounding with the other components of the personal care composition). With a pre-swollen elastomer, the weight percentages recited above are of the elastomer particles alone (excluding the weight of the solvent). Furthermore, the non-emulsifying silicone elastomer may be supplied as a powder. It is recognized that powdered non-emulsifying silicone elastomers may swell in the personal care composition when in the presence of non-polar oils or other suitable fluids. In certain embodiments, the first and second non-emulsifying silicone elastomer may be a pre-swollen elastomer and a powder, respectively or vise versa.

In particular embodiments, the personal care composition may comprise from about 10% to about 70%, and alternatively 30% to about 50% of one or more solvents. Suitable solvents include non-polar oils. The non-polar oils may be natural, synthetic, saturated, unsaturated, straight chained, branched chained, linear, cyclic, aromatic, volatile, non-volatile, and combinations thereof. Non-limiting examples of suitable non-polar oils include silicone oils, hydrocarbon oils, and mixtures thereof. Examples of suitable non-polar hydrocarbon oils include but are not limited to mineral oils and branched chain hydrocarbons (such as commercially available, for example, under the tradenames Permethyl™ (Permethyl Corporation™) and Isopar™ (Exxon™)). Examples of suitable non-polar silicone oils include but are not limited to linear and cyclic polydimethylsiloxanes, including cyclomethicones (cyclotetrasiloxane, cyclopentasiloxane, cyclohexasiloxane), dimethicones, and mixtures thereof. Commercially available examples of these types of silicones include the Dow Corning 200 series, Dow Corning 344, and Dow Corning 345 (all available from Dow Corning™ Corp.); and SF1202, SF1204, and the Viscasil™ series (all available from the G.E. Silicones™). Additional non-polar silicone oils include alkyl (for example, 2 carbons to 30 carbons) and aryl (for example, phenyl or styrenyl) substituted silicones, including by not limited to phenyl methicone, phenyl dimethicone, phenyl trimethicone, diphenyl dimethicone, phenylethyl dimethicone, hexyl dimethicone, lauryl dimethicone, cetyl dimethicone, stearyl dimethicone, bis-stearyl dimethicone, and mixtures thereof. In certain embodiments the non-polar emollient is low viscosity, meaning a viscosity less than 50 cst. Many of the aforementioned solvents may also be used as an emollient in addition to or independent of use as a solvent for the non-emulsifying silicone elastomers.

Emulsifier—In one aspect, the composition comprises one or more emulsifiers that may be linear, branched, and/or cross-linked. In certain embodiments, the personal care composition may comprise from about 0.05% to about 20%, or from about 0.1% to about 10%, by weight of the composition, of total emulsifier. Non-limiting examples of emulsifiers are disclosed in U.S. Pat. No. 3,755,560, U.S. Pat. No. 4,421,769, and McCutcheon's Detergents and Emulsifiers, North American Edition and International Edition, pages 235-246 (1993).

Emulsifiers may include emulsifying silicone elastomers. Suitable emulsifying silicone elastomers include cross-linked organopolysiloxane elastomers having at least one polyalkyl ether unit. These cross-linked elastomers may also be co-modified to include alkyl substituents. Particularly useful emulsifying polyoxyethylene cross-linked elastomers include Shin Etsu's KSG-21, KSG-210, KSG-24, KSG-240, KSG-31, KSG-310, KSG-32, KSG-320, KSG-33, KSG-330, KSG-34, and KSG-340.

Linear or branched type silicone emulsifiers are also useful in this application. Particularly useful polyether or polyglycerolated modified silicone emulsifiers include Shin Etsu's KF-6100, KF-6104, KF-6105, KF-6011, KF-6012, KF-6013, KF-6015, KF-6016, KF-6017, KF-6043, KF-6028, and KF-6038.

The personal care composition may optionally comprise particulate material, insoluble sunscreens, and/or skin care actives,

Particulate Materials—In particular embodiments, the composition comprises from about 0.1% to about 40%, or from about 1% to about 30%, or from about 5% to about 20%, by weight of the composition, of one or more particulate materials. Non-limiting examples of suitable powders include inorganic powders (for example, iron oxides, titanium dioxides, zinc oxides, silica), organic powders, composite powders, optical brightener particles, and mixtures of any of the foregoing. These particulates can, for instance, be platelet shaped, spherical, elongated or needle-shaped, or irregularly shaped; surface coated or uncoated; porous or non-porous; charged or uncharged; and can be added to the current compositions as a powder or as a pre-dispersion. In one embodiment, the particulate material is hydrophobically coated.

Suitable organic powder particulate materials include, but are not limited, to polymeric particles chosen from the methylsilsesquioxane resin microspheres, e.g., Tospearl™ 145A, (Toshiba Silicone); microspheres of polymethylmethacrylates, e.g., Micropearl™ M 100 (Seppic); the spherical particles of crosslinked polydimethylsiloxanes, e.g., Trefil™ E 506C or Trefil™ E 505C (Dow Corning Toray Silicone); spherical particles of polyamide, e.g., nylon-12, and Orgasol™ 2002D Nat C05 (Atochem); polystyrene microspheres, e.g., Dyno Particles, sold under the name Dynospheres™, and ethylene acrylate copolymer, sold under the name FloBead™ EA209 (Kobo); aluminum starch octenylsuccinate, e.g., Dry Flo™ (National Starch); polyethylene particulates, e.g., Microthene™ FN510-00 (Equistar) and Micropoly® 220L (Micro Powders, Inc.); microspheres of polypropylene, e.g., Mattewax™ 511 (Micro Powders, Inc.); silicone resin; polymethylsilsesquioxane silicone polymer; platelet shaped powder made from L-lauroyl lysine; and mixtures thereof.

In particular embodiments, the composition comprises interference pigments, including hydrophobically-modified interference pigments. Herein, “interference pigments” means thin, plate-like layered particles having two or more layers of controlled thickness. The layers have different refractive indices that yield a characteristic reflected color from the interference of typically two, but occasionally more, light reflections, from different layers of the plate-like particle. One example of interference pigments are micas layered with about 50-300 nm films of TiO₂, Fe₂O₃, silica, tin oxide, and/or Cr₂O₃ and include pearlescent pigments. Interference pigments are available commercially from a wide variety of suppliers, for example, Rona (Timiron™ and Dichrona™), Presperse (Flonac™), Englehard (Duochrome™), Kobo (Interfine, Interval, SK-45-R, and SK-45-G), BASF (Sicopearls™) and Eckart (Prestige™). In one embodiment, the average diameter of the longest side of the individual particles of interference pigments is less than about 75 microns, and alternatively less than about 50 microns.

Particulates may also include colorants. Non-limiting examples of suitable colorants include iron oxides, ferric ammonium ferrocyanide, manganese violet, ultramarine blue, and chromium oxide, phthalocyanine blue and green pigment, encapsulated dyes, inorganic white pigments, for example TiO₂, ZnO, or ZrO₂, FD&C dyes, D&C dyes, and mixtures thereof.

Insoluble Sunscreens—In particular embodiments, the composition comprises from about 0.001% to about 10%, or from about 0.1% to about 5%, by weight of the composition, of an insoluble sunscreen. Non-limiting examples of suitable insoluble sunscreens include methylene bis-benzotriazolyl tetramethylbutyl-phenol (Tinosorb M), titanium dioxides, zinc cerium oxides, zinc oxides, and derivatives and mixtures thereof.

Skin Care Actives—In particular embodiments, the composition comprises one or more skin care actives. Suitable skin care actives include, but are not limited to, vitamins, peptides, sugar amines, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, and antifungals. These skin care actives and others are provided in further detail in U.S. Application Publication Nos. US2006/0275237A1, US2004/0175347A1, and US2006/0263309A1.

Particularly suitable skin actives include a vitamin B₃ compound, a sugar amine, a peptide, a hexamidine compound, and combinations thereof. As used herein, “vitamin B₃ compound” means a compound having the formula:

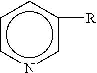

wherein R is —CONH₂ (i.e., niacinamide), —COOH (i.e., nicotinic acid) or —CH₂OH (i.e., nicotinyl alcohol); derivatives thereof; and salts of any of the foregoing. As used herein, “sugar amine” includes isomers and tautomers of such and its salts (e.g., HCl salt) and its derivatives. Examples of sugar amines include glucosamine, N-acetyl glucosamine, mannosamine, N-acetyl mannosamine, galactosamine, N-acetyl galactosamine, their isomers (e.g., stereoisomers), and their salts (e.g., HCl salt). As used herein, “peptide” refers to peptides containing ten or fewer amino acids and their derivatives, isomers, and complexes with other species such as metal ions (e.g., copper, zinc, manganese, magnesium, and the like). As used herein, “hexamidine compound” means a compound having the formula:

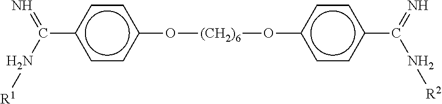

wherein R¹ and R² are optional or are organic acids (e.g., sulfonic acids, etc.), and its salts and derivatives.

Further suitable skin care actives include white tea extract, green tea extract, ginseng, and other natural or botanical compounds.

### Methods

The present invention further relates to methods of protecting keratinous tissue from the harmful effects of UV radiation. Such methods generally involve attenuating or reducing the amount of UV radiation which reaches the keratinous tissue. In certain embodiments, the personal care compositions described herein are suitable for use as a sunscreen to provide protection to keratinous tissue from the harmful effects of UV radiation such as sunburn, dry or damaged hair, and premature aging of the skin.

The personal care compositions of the present invention may regulate and/or improve the condition of keratinous tissue, non-limiting examples of which include reducing the appearance of wrinkles, reducing the appearance of deep lines, reducing the appearance of fine lines, reducing the appearance of large pores, reducing the thickness of keratinous tissue, increasing the convolution of the dermal-epidermal border, increasing elasticity, reducing the appearance of cellulite, reducing the appearance of discoloration, reducing the appearance of hyperpigmentation, reducing the appearance of under-eye circles, reducing the appearance of sallowness, and combinations thereof.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. After application, the composition may be allowed to remain on the keratinous tissue.

### EXAMPLES

The following describe non-limiting examples of the personal care composition. The reported percentages indicate the weight of the component expressed as a percentage of the total weight of the personal care composition. Each Example may comprise one or more of the optional ingredients in amounts as disclosed herein. The Examples may be prepared as follows.

In a suitable vessel, combine the sunscreens (e.g., ethylhexyl salicylate, homosalate, octocrylene, butyl methoxydibenzoylmethane, benzophenone-3), sunscreen solvent (e.g., isopropyl lauroyl sarcosinate), and wax (e.g., Cirebelle 303, Accumelt 82, or Accumelt 90) and heat to 90° C. with mixing. When the solution is translucent and free of particulates to the naked eye, add the remaining ingredients to the same container. Stir vigorously until smooth and homogeneous while maintaining the temperature of the product above the wax melt point. Remove the product from the heat source and pour the hot formulation into suitable containers and allow it to cool to room temperature without vibration or disturbance.

| Ingredients | Ex. 1 | Ex. 2 | Ex. 3 | Ex. 4 | Ex. 5 |
|---|---|---|---|---|---|
| Ethylhexyl Salicylate | 4.00 | 5.00 | 4.50 | 4.50 | 4.50 |
| Homosalate | 1.00 | 15.00 | 9.00 | 9.00 | 9.00 |
| Octocrylene | 1.20 | 2.70 | 2.10 | 2.10 | 2.10 |
| Butyl Methoxy- | — | 6.00 | 2.50 | 2.50 | 2.50 |
| dibenzoylmethane | | | | | |
| Benzophenone-3 | 2.00 | 3.00 | 3.10 | 3.10 | 3.10 |
| Isopropyl Lauroyl | 3.00 | 5.00 | 3.00 | 3.00 | 3.00 |
| Sarcosinate | | | | | |
| Dow Corning ™ | — | qs | qs | qs | qs |
| 9045¹ | | | | | |
| Dow Corning ™ | qs | — | — | — | — |
| 9040¹ | | | | | |
| Dow Corning ™ | — | — | 5.00 | 5.00 | — |
| 9701² | | | | | |
| Dow Corning ™ | 7.00 | — | — | — | 5.00 |
| 9560³ | | | | | |
| KSG-310⁴ | 3.00 | 3.00 | 3.00 | 3.00 | 3.00 |
| Tospearl ™ 145A⁵ | 3.00 | 9.00 | 9.00 | 8.00 | 10.00 |
| Micropoly 220L⁶ | 3.00 | 9.00 | 9.00 | 7.00 | 5.00 |
| Cirebelle 303⁷ | 4.00 | 8.00 | 7.00 | — | — |
| Accumelt 72⁸ | — | — | — | — | 5.00 |
| Accumelt 90⁸ | — | — | — | 5.00 | — |
| KF-6105⁹ | — | — | — | — | 0.1 |
| KF-6038¹⁰ | — | — | — | — | 0.1 |
| Vitamin E acetate | — | — | 0.5 | — | — |
| Perfume | 0.3 | 0.3 | 0.3 | 0.3 | 0.3 |
| Total | 100.00 | 100.00 | 100.00 | 100.00 | 100.00 |

¹Dimethicone Crosspolymer (about 12%) and Cyclopentasiloxane (about 88%) from Dow Corning ™ Midland, MI.
²Dimethicone/Vinyl Dimethicone Crosspolymer/Amorphous fumed silica from Dow Corning ™ Midland, MI.
³Dimethicone/Vinyl Dimethicone Crosspolymer from Dow Corning ™, Midland, MI.
⁴PEG-15/Lauryl Dimethicone Crosspolymer and Mineral Oil from Shin-Etsu ™,
Newark, CA.
⁵Polymethylsilsesquioxane from Momentive ™ Performance Materials, Inc., Albany, NY.
⁶Polyethylene from Micro Powders, Inc., Tarrytown, NY.
⁷Polyethylene Wax from Arch Chemicals, Inc., South Plainfield, NJ.
⁸Synthetic Wax from The International Group Inc., Toronto, Ontario.
⁹Lauryl Polyglyceryl-3 Polydimethylsiloxyethyl Dimethicone from Shin-Etsu ™,
Newark, CA.
¹⁰Lauryl PEG-9 Polydimethylsiloxyethyl Dimethicone from Shin-Etsu ™, Newark, CA.

### COMPARATIVE EXAMPLES

| | | Hardness | Spreadability |
|---|---|---|---|
| | Product | (g) | (kg) |
| A | Neutrogena ® pure & free baby | 298 | 7.0 |
| | sunblock stick¹¹ | | |
| B | Banana Boat ® Sport Sunscreen Stick¹² | 225 | 6.8 |
| C | Covergirl CG Smoothers ® Concealer | 214 | 7.0 |
| | Corrector Stick¹³ | | |
| D | Dermatone Spot Protection Stick¹⁴ | 186 | 6.8 |
| E | Ex. 3 (within scope of invention) | 35 | 2.5 |
| F | Olay total effects plus SPF 15, | <1 | 0.1 |
| | fragrance free¹⁵ | | |

¹¹SPF 60+, Sunscreens: 6% titanium Dioxide, 4% zinc oxide; Waxes: beeswax, cetyl alcohol, candelilla wax, ozokerite, paraffin, polyethylene, steric acid.
¹²SPF 30, Sunscreens: 7.0% homosalate, 7.5% ethylhexyl methoxycinnamate, 5.0% ethylhexyl salicylate, 8.0% octocrylene, 6.0% benzophenone-3; Waxes: ozokerite, isocetyl alcohol, beeswax, cocoa butter
¹³No SPF protection, Waxes: candelilla wax, ozokerite, carnauba wax
¹⁴SPF 23, Sunscreens: 8.0% padimate O, 4.0% benzophenone-3; Waxes: paraffin, cocoa butter
¹⁵SPF 15, Sunscreens: 4.0% ethylhexyl salicylate, 2.0% butyl methoxydibenzoylmethane, 1.0% phenylbenzimidazole sulfonic acid, 1.0% octocrylene; Waxes: stearyl alcohol, cetyl alcohol, beheny alcohol

Hardness is a measure of a material's resistance to deformation when a force is applied. In contrast to the hardness measurement methodology, the cone shaped apparatus used to determine spreadability offers no locations into which the product can be packed or compressed. Thus, when determining spreadability the product is forced to flow outward and maximum force required to achieve this flow measured.

In the comparative examples, product forms sold as sticks (many of which contain sunscreens) all have high hardness values and correspondingly high spreadability values as well, indicating that the sticks are difficult to spread uniformly on skin. Poor spreadability then translates to poor sunscreen efficiency in true consumer use situations. Alternatively, spreadability is low for traditional creams, boosting sunscreen efficiency on skin, but with the low product hardness the form is not unique. The personal care composition of the present invention delights consumers by attaining a noticeable hardness without compromising spreadability offers the advantage of consumer delight and sunscreen performance.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable personal care composition in the form of a substantially anhydrous oil matrix comprising:
a) from about 1% to about 10% of a hydrocarbon wax;
b) greater than about 10% of a polar oil;
c) from about 1% to about 30% of a first non-emulsifying silicone elastomer;
wherein the weight ratio of wax to polar oil is from about 0.01 to about 0.5;
wherein the personal care composition exhibits a spreadability of 1.0 kg to about 5.0 kg and a hardness of about 3 g to about 120 g.

2. The stable personal care composition of claim 1, wherein the hydrocarbon wax is selected from the group consisting of polyethylenes, polymethylenes, and combinations thereof.

3. The stable personal care composition of claim 1, wherein the hydrocarbon wax comprises from 20 to 100 carbon atoms.

4. The stable personal care composition of claim 1, wherein the hydrocarbon wax is a straight-chain hydrocarbon wax comprising less than 5% CHR₃ polymeric subunits, wherein R is a hydrocarbon chain of length C₁-C₁₀₀,

5. The stable personal care composition of claim 1, wherein the hydrocarbon wax comprises a polydispersity index less than about 1.1.

6. The stable personal care composition of claim 1, wherein the hydrocarbon wax comprises a melt point of from about 55 degrees to about 150 degrees C.

7. The stable personal care composition of claim 1, wherein the polar oil comprises a sunscreen.

8. The stable personal care composition of claim 7, wherein the sunscreen is selected from the group consisting of benzophenone-3, bis-ethylhexyloxyphenol methoxyphenyl triazine, butyl methoxydibenzoylmethane, diethylamino hydroxy-benzoyl hexyl benzoate, ethylhexyl methoxy-cinnamate, ethylhexyl salicylate, ethylhexyl triazone, octocrylene, homosalate, polysilicone-15, and mixtures thereof.

9. The stable personal care composition of claim 1, wherein the polar oil is selected from the group consisting of butyl phthalimide, isopropyl phthalimide, phenylethyl benzoate, dicaprylyl carbonate, isopropyl lauroyl sarcosinate, butyl octylsalicylate, dioctyl malate, dicaprylyl maleate, di-isopropyl adipate, isononyl isononanoate, isopropyl isostearate, propylene glycol dicaprate, C12-15 alcohol benzoate, derivatives, and mixtures thereof.

10. The stable personal care composition of claim 1, wherein the polar oil comprises:
a) a sunscreen selected from the group consisting of benzophenone-3, bis-ethylhexyloxyphenol methoxyphenyl triazine, butyl methoxydibenzoyl-methane, diethylamino hydroxy-benzoyl hexyl benzoate, ethylhexyl methoxy-cinnamate, ethylhexyl salicylate, ethylhexyl triazone, octocrylene, homosalate, polysilicone-15, and mixtures thereof; and
b) a member selected from the group consisting of butyl phthalimide, isopropyl phthalimide, phenylethyl benzoate, dicaprylyl carbonate, isopropyl lauroyl sarcosinate, butyl octylsalicylate, dioctyl malate, dicaprylyl maleate, di-isopropyl adipate, isononyl isononanoate, isopropyl isostearate, propylene glycol dicaprate, C12-15 alcohol benzoate, derivatives, and mixtures thereof.

11. The stable personal care composition of claim 1, further comprising a skin-care active, wherein the skin-care active is selected from the group consisting of vitamins, peptides, sugar amines, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, antifungals, and mixtures thereof.

12. The stable personal care composition of claim 1, further comprising a skin-care active, wherein the skin-care active is selected from the group consisting of a vitamin B₃ compound, a sugar amine, a peptide, a hexamidine compound, and combinations thereof.

13. The stable personal care composition of claim 1, further comprising a particulate material.

14. The stable personal care composition of claim 1, further comprising from about 1% to about 10% of a second non-emulsifying silicone elastomer.

15. The stable personal care composition of claim 14, wherein the first non-emulsifying silicone elastomer and the second non-emulsifying silicone elastomer are either pre-swollen or powdered, provided that the first non-emulsifying silicone elastomer and second non-emulsifying silicone elastomer are not both pre-swollen or powdered.

16. The stable personal care composition of claim 1, wherein the personal care composition is a beauty care composition.
